(19) United States
(12) Patent Application Publication
(10) Pub. No.: US 2006/0148767 A1
(43) Pub. Date: Jul. 6, 2006

(54) Use of acyl salicylates as heat shock inducers

(76) Inventors: George Cioca, Lake Grove, NY (US); Mosa Rossi, Naples (IT); Marlo Derosa, Naples (IT)

Correspondence Address: Karen A Lowney;Estee Lauder, 125 Pinelawn Road, Melville, NY, 11747, US

(21) Appl. No.: 10/496,470

(22) PCT Filed: Dec. 6, 2002
(86) PCT No.: PCT/US02/39150

§ 371 Date: Jun. 29, 2005

(30) Foreign Application Priority Data
Dec. 6, 2001 (IT) MI2001A002573

(51) Int. Cl. A61K 31/60; A61K 8/37

(52) U.S. Cl. 514/159; 424/59

(57) ABSTRACT

The invention relates to acyl-salicylates as heat shock response inducers and the use thereof as active ingredients in cosmetic and/or pharmaceutical preparations.

### FIELD OF THE INVENTION

The present invention relates to acyl-salicylates as heat shock response inducers and the use thereof as active ingredients in cosmetic and/or pharmaceutical preparations.

### BACKGROUND OF THE INVENTION

Homeostasis is the preservation of the chemical architecture and of the functional properties of a cell or an organism under stress conditions. A feature of homeostasis is the rapid expression of genes whose products are specifically dedicated to protect cellular components and functions from stress. One of the best known mechanisms for cell protection against stress is the heat shock response, which results in the expression of heat shock proteins (Ann. Rev. Biochem. 55, 1151-1191, 1986; Ann. Rev. Genet. 22, 631-677, 1988). During aging, cells gradually lose their ability to synthesize the proper amounts of heat shock proteins and defend themselves from cellular damage. Among heat shock proteins, chaperonins are one of the most important class, since they prevent the incorrect association within and between polypeptide chains during folding of newly synthesized proteins and protect the pre-existing proteins under cellular stresses (Trends Biochem. Sci. 19, 20-25, 1994). Chaperonins function also in the absence of stress, namely under normal physiological conditions, helping cellular proteins to fold correctly during synthesis on ribosomes.

From the most recent data reported in literature, it appears that cellular membranes act as sensors to the external factors inducing stress and that the modification of their physical state influences the expression of heat shock genes. The membrane bilayer has fluidity properties that permit the cell to sense changes in temperature, pH, osmotic and atmospheric pressure etc. For example, increases in temperature activate, in the cells, the biochemical mechanisms that cause an increase in the intrinsic viscosity of the membrane to compensate the increase in fluidity induced by temperature (Proc. Natl. Acad. Sci. USA 23, 3870-3875, 1996). In particular, evidence is now accumulating that membrane lipids may participate, as molecular chaperones, in the folding and unfolding of membrane proteins (J. Biol. Chem. 274, 36827-36830, 1999). Recent reports suggest that modulation of the membrane's physical state influences the expression of heat shock genes in the cell by lowering the temperature at which the heat shock response occurs.

From the above considerations, it becomes apparent that compounds able to induce a heat shock response can create a preventive defense in the cells and protect them during aging and other stress-related conditions. Indeed, it is well established in the literature that the over-expression of one or more heat shock proteins can be sufficient to protect cells and tissues against otherwise lethal exposure to diverse environmental stresses including hydrogen peroxide and other oxidants, toxic chemicals, extreme temperatures, and ethanol-induced toxicity. (Parsell and Lindquist, in The Biology of Heat Shock Proteins and Molecular Chaperones, Morimoto et al., eds., 457-494, Cold Spring Harbor Laboratory Press, 1994). Cell membrane fluidity has also been clearly associated with the induction. Drugs such as chlorpromazine, procaine, dibucaine, which are used as psychotropics or local anesthetics, are acknowledged to produce stress genes induction, since they interact with membranes causing their deformation or fluidification (J. Invest. Dermatol. 104, 448456, 1995). This mechanism occurs, also, in the case of a series of hydroxylamine derivatives, such as Bimoclomol (U.S. Pat. No. 5,296,606; EP 1020187A), that has been found to enhance the accumulation of heat-shock proteins in mammalian cells, to protect them from damages induced by stress, to prevent and repair skin damage on mice exposed to ultraviolet B irradiation and to accelerate wound healing in diabetic rats (Nature Medicine 3, 1150-1154, 1997).

As regards the skin, Maytin has reported (J. Invest. Dermatol. 104, 448, 1995) that heat shock proteins play an important role in the protection of the skin from environmental stresses and participate in the prevention and repair of damages caused by exposure to light, heat, chemical injuries, and other traumas. In addition, Polla (Dermatologica 180, 113, 1990) has suggested that UV-B irradiation induces heat shock response, that protects human skin from cell damage and is part of the natural defenses that follow exposure to solar radiation.

It is, also, well established that stress proteins are crucial for the maintenance of cell health and integrity in some patho-physiological conditions that involve other tissues or organs, such as cardiomyopaties, ischemia, amyotropic lateral sclerosis and Alzheimer's, Parkinson's and Huntington's diseases (J. Mol. Cell. Cardiol. 27, 45-52, 1995; J. Clin. Invest. 95, 1446-1456, 1995; Nature Medicine 3, 1150-1154, 1997, EP1020187 A).

As the foregoing summary shows, it is unequivocal that normal production of heat shock proteins by cells, as well as their production in response to various sources of stress, provide a substantial protective effect on cells and tissues in the body. It also appears that a large number of pathological conditions are associated with stressed cells and/or the lack or inadequacy of an effective heat shock response, and therefore can benefit from the induction of a heat shock response. There thus continues to be a strong therapeutic need for compounds that are capable of inducing the heat shock response in cells, both to protect the cells from future assault and to assist in repairing damage already done to cells exposed to stress. It has now been found that acyl salicylate derivatives or the salts thereof are able to induce a strong heat shock response.

### SUMMARY OF THE INVENTION

The invention relates to a method of inducing the production of heat shock proteins in a cell which comprises applying to the cell an induction-effective amount of a C3-C25 acyl salicylate. In practical application, the invention also relates to a method of treating or alleviating a disease condition associated with a heat shock response which comprises administering to an individual in need of such treatment or alleviation an induction effective amount of a C3-C25 acyl salicylate. The invention also relates to a method of preventing, alleviating or treating damage to cells or tissues exposed to environmental stress which comprises administering to the cells or tissue an induction effective amount of a C3-C25 acyl salicylate. Preferably, the cells treated are skin cells. The invention also relates to a method of preventing damage due to shock in living tissue or organs, such as those intended for transplant, which comprises treating the tissue or organs with an induction effective amount of a C3-C25 acyl salicylate. The invention also relates to pharmaceutical or cosmetic compositions comprising induction effective amounts of a C3-C25 acyl salicylate.

### BRIEF DESCRIPTION OF THE FIGURE

FIG. 1 illustrates the detoxification effect of 5 μM (Series 1) and 50 μM (series 2) doses of decanoyl salicylate (1), acetyl salicylate (2) and salicylic acid (3). The values represent the increase in cell viability relative to the control.

### DETAILED DESCRIPTION OF THE INVENTION

The acyl salicylates employed in the method of the present invention are acyl-salicylic acids, and salts thereof, particularly pharmaceutically or cosmetically acceptable anionic salts, wherein the acyl group is a C₃-C₂₅ straight or branched acyl, preferably C₃-C₁₂ acyl, which may be saturated or unsaturated, and/or substituted with hydroxyl, carboxyl, or carbonyl groups. As used in the present specification and claims, the term “acyl salicylates” shall be understood to refer to any of the forgoing compounds. “Induction effective amounts” of the acyl salicylates are those amounts that are capable of increasing the amount of heat shock proteins produced by a cell at least about 10%, preferably at least about 25%, more preferably at least about 50%, relative to the amount produced by an untreated control cell. The amounts used in practical application of formulation are discussed in greater detail below.

Particularly preferred acyl-salicylic acids are obtained by acylation of salicylic acid with C₃-C₁₂ straight aliphatic acids. The compounds of the present invention can be prepared from salicylic acid by reaction with the appropriate acid anhydride or chloride, such as is described in the present examples 1 and 2.

The connection of certain salicylates with a heat shock response has been previously mentioned. For example, sodium salicylate (2-30 mM) has been shown to promote the activation of DNA binding by heat shock transcription factor (HSF), and extends the period during which HSF is maintained in the active state; however, the compound does not induce heat shock gene transcription (Jurivich et al., Science 225: 1243-1245, 1992). Aspirin (acetyl salicylate) when administered at 0.4 mM to erythroleukemic cells during or after a hyperthermic treatment causes an increase in the amount of HSP70 synthesized and prolongs the time of synthesis (Amici et al., Cancer Research 55: 4452-4457, 1995). However, this pattern of activity suggests that aspirin only affects the heat shock response in connection with a stress condition, and not on its own The activity of the longer chain acyl salicylates is nonetheless quite surprising on a number of grounds. The capability of some acyl salicylates, particularly propanoyl, n-butanoyl, n-octanoyl and n-decanoyl salicylates, to induce heat shock response has been tested in three different in vitro systems. In one case (example 3), the compounds were found to substantially increase HSP70 production in the absence of any heat shock. In another, when these compounds are tested for their effect on heat shock induction in fibroblasts, and the results compared with the data reported in literature for Bimoclomol, an well-known heat shock inducer, the longer chain acyl salicylates out-performed the known compound, inducing heat shock response at lower temperatures, while substantially no increase was observed with acetyl salicylate (see example 4). In a third test, the ability of various acyl salicylates to actually provide fibroblast cells protection against cytotoxic proteins was evaluated; not only was the longer chain acyl salicylate capable of protecting the cells and increasing cell viability, but the effect that was observed was greater than that observed with acetyl salicylate and salicylic acid. The overall observations in all tests show that, when acyl salicylates are added at 5 μM doses to the cultured cells in the absence of heat stress, the basal level of heat shock proteins is on the average doubled.

It is well established that the production of heat shock protein in vivo provides substantial protection or defense against environmental assaults or pathological conditions. For example, it has been demonstrated that transgenic mice overexpressing HSP-70 are significantly more resistant to ischemia relative to normal mice (Marber et al. J. Clin. Invest. 95: 1446-1456, 1995). Similarly, known heat shock response inducers have been stated as having beneficial effects on diabetes, neuropathies, angiopathies, and the like (U.S. Pat. No. 5,296,606). In addition, it is well documented that alterations in the heat shock protein levels are observed in association with several disease conditions, such as cardiomyopathies, or Alzheimer's disease. (Welch, Physiological Review 72: 1063-1081, 1992; Thomas et al., TIBS 20: 4456-4459, 1995; Marimoto and Santoro, Nature Biotech 16: 833-838, 1998). It is also known that cellular levels of heat shock protein decreases generally with age. It is therefore understood that in one embodiment of the invention, the acyl salicylates can be used to treat, alleviate or prevent diseases or conditions associated with a reduction or lack of production of heat shock proteins, or in which heat shock induction is known to be an important protective factor. Since the data provide herein demonstrate that acyl-salicylates increase the natural defense mechanism of the cells in terms of heat shock protein production, they may be considered as valid active principles for the preparation of medicaments for the prevention and treatment of pathologies or conditions in which heat shock induction is important such as Huntington's, Parkinson's and Alzheimer's diseases, wound healing and cardiovascular diseases. As used in the present specification and claims, such diseases and conditions shall be referred to collectively as “heat shock-related conditions.”

For the treatment of the conditions described above, the compounds of the present invention will be administered in any fashion appropriate to the condition to be treated. Frequently, the preferred mode of administration will be oral, preferably in the form of pills, tablets or granulates, formulated according to conventional methods and mixed with suitable excipients. However, where appropriate parenteral administration of the compositions can also be employed, e.g., intramuscular, intraperitoneal, intravenous, subcutaneous, and the like. The acyl-salicylates can be contained in such pharmaceutical formulations at concentrations between 0.1 and 90% w/w, preferably between 1 and 20% w/w. The formulation, vehicles, and mode of administration of compositions of this type are well known in the art, and examples of such can be found in Remington's Pharmaceutical Sciences, 18^{th} edition, 1990, the contents of which is incorporated herein by reference.

The acyl-salicylates are able to activate cytoprotective responses by increasing the ability of the cells to efficiently respond to different stress conditions. They have been proven more effective than Bimoclomol that, in the described experiments, induces heat shock response only at higher temperatures, and under more severe condition of stress. Because they are able to induce heat shock proteins even under conditions that would not necessarily produce a heat shock response, the acyl salicylates are useful not only in the treatment of existing stress-related conditions, but also in preventing the occurrence of damage by application before exposure to stress. Considering that the skin is the body part most exposed to environmental stress, the acyl salicylates are particularly preferred for the treatment and prevention of topical or skin conditions that are associated with a heat shock response. One example of such a use is in the promotion of wound healing. As example 6 herein shows, application of the acyl salicylates to wounded skin has the capacity to enhance the progress of wound closure. Thus the acyl salicylates can be used to treat any type of surface wound, for example, ulcers, lacerations, diabetic ulcers, burns, trauma, inflammatory lesions, stasis ulcers, periodontal conditions, surgical wounds and other such conditions. It will be recognized as well that, although the term “wound” is routinely associated with a skin disruption, the acyl salicylates can also be used to enhance healing of internal wound tissue such as intraperitoneal tissue damaged in the course of surgery. In the present context, then, “wound healing” will be understood to encompass both internal and external wounds.

In a particularly preferred embodiment, the acyl salicylates are used to prevent and alleviate skin damage associated with UV radiation. Exposure to UV radiation has been unequivocally shown to be associated with an increase in the production of heat shock protein (Brunet and Giacomoni, Mutat. Res. 219: 217-224, 1989), presumably providing a cytoprotective effect to exposed cells. As further shown herein, in Example 5, the acyl salicylates do provide a protective effect against UV radiation exposure. Therefore, the acyl salicylates can be used as a protective agent, being applied in advance of sun or other anticipated UV exposure, to prevent erythema and any other skin damage associated with exposure to UV. The acyl salicylates can also be applied during or after UV exposure to enhance the skin's natural heat shock response and alleviate or reduce skin damage that has already been initiated. The treatment can also be applied to skin exposed, or expected to be exposed to other environmental insults that provoke a skin response, such as tobacco smoke, air pollution, oxidative stress, and harsh chemical exposure. As used herein, these factors will all collectively be referred to as “environmental stress”.

In another preferred embodiment, the acyl salicylates are used to treat, alleviate and prevent the effects of aging on skin cells. As noted above, it is known that levels of heat shock proteins decrease in aging cells. As shown in Examples 2 and 3, acyl salicylates are capable of enhancing the production of heat shock protein in skin cells, in fact, at a better rate than that observed with the best known heat shock inducer, Bimoclomol. Thus, acyl salicylates can be used to enhance heat shock proteins in aging skin cells, with the benefit of reducing the damaging effects of aging on such cells. This is equally applicable to skin cells that are subject to photoaging as well as chronological aging. Expected effects of the treatment of aging skin cells are enhancement of elastin and collagen production, prevention or retardation of skin atrophy, prevention or reduction of skin thinning, and the like.

Topical compositions for pharmaceutical use are described in Remington's Pharmaceutical Sciences, supra. Guidance on formulation for cosmetic use can also be found in Harry's Cosmeticology, 8^{th} edition, M. Reiger, Ed. 2000, the contents of which are incorporated herein by reference. Typically, these compositions will be in form of oil, cream, gel, powder, emulsions, suspensions and the like, and will contain acyl-salicylates in amounts comprised between 0.1 and 50% w/w, preferably between 0.5 to 10% w/w, more preferably between 1 and 5% w/w.

It may be desirable to combine the acyl salicylates of the invention with other active materials in formulation. Examples of other actives that may be added to the acyl salicylate formulation include, but are not limited to, those that improve or eradicate age spots, keratoses and wrinkles, analgesics, anaesthetics, anti-acne agents, antibacterials, antiyeast agents, antifungal agents, antiviral agents, antidandruff agents, antidermatitis agents, antipruritic agents, antiemetics, anti-inflammatory agents, antihyperkeratolytic agents, anti-dry skin agents, antiperspirants, antipsoriatic agents, antiseborrheic agents, antiaging agents, antiwrinkle agents, antiasthmatic agents and bronchodilators, sunscreen agents, antihistamine agents, skin lightening agents, depigmenting agents, wound-healing agents, vitamins, corticosteroids, tanning agents, or hormones.

In general, the compositions of the invention can be used and administered in a number of ways. For example, because of the overall benefit of the acyl salicylates to cell health with little or no adverse effect on normal cellular metabolism, a chronic administration or application may be desired, particularly when an individual is subject continually subject to certain risk factors, such as aging or pathological conditions. By “chronic administration or application” is meant that the period of administration or application may be over the lifetime of the user, preferably for a period of at least about one month, more preferably from about three months to about twenty years, more preferably from about six months to about ten years, more preferably still from about one year to about five years. On the other hand, an acute administration or application may be preferred, in anticipation of, during or after exposure to a environmental stress condition, such as UV radiation. In many such cases, the compositions will be used on an as-needed basis, although, in the case of certain stresses, such as routine exposure to pollution or UV, a chronic preventive application may be preferable. Given the guidance provided herein, the appropriate uses of the compositions will be readily apparent to one skilled in the art.

A further application field of the compounds of the present invention is the use thereof in the preservation of cellular systems, organs or part thereof, for use in transplants. In fact the organ, before its transplant, undergoes a series of shocks due to the non-physiological storage environment. The presence of acyl-salicylates in the medium would improve preservation of the organ, by activating the cells defense and repair mechanisms. The acyl-salicylates will be used in the preparation of preserving solutions for cellular systems, organs or tissues, optionally mixed with other compounds suitable for the preservation of biological materials. The compounds of the invention for this purpose will be present in such solutions at concentrations from 0.1 to 50% w/w, preferably from 1% to 10% w/w.

The following examples further illustrate the invention.

### EXAMPLES

### Example 1

### Synthesis of the Acyl Salicylate Derivatives from the Acid Anhydrides: Synthesis of Propanoyl Salicylate

1 Equivalent of salicylic acid was dissolved in 3.0 equivalents of propanoyl anhydride in the presence of catalytic amounts of concentrated sulfuric acid. The mixture was stirred at room temperature for 30 minutes and kept at 70° C. for 2 hours. The reaction mixture was poured into ice water and the precipitated product was filtered in vacuo, dried and recrystallized tree times from toluene or ethanol as solvent. The purified propanoyl salicylate has melting point of 96-98° C. and the UV-visible spectrum shows a characteristic peak at 276 nm. The ¹H-NMR spectrum of the synthesized propanoyl salicylate is consistent with the proposed structure. The spectrum shows in the correct integration and multiplicity the proton signals of the propanoyl chain (methyl, triplet, 1.28 ppm, 3 protons; methylene, quartet, 2.65 ppm, 2 protons) and of the aromatic ring (H-1, doublet, 8.11 ppm, 1 proton; H-2 and H-3, two double doublets, 7.32 and 7.61 ppm, 1+1 protons, H4, doublet, 8.10 ppm, 1 proton). The stability of the propanoyl salicylate both as crystalline compound and in solution is compatible with its use in cosmetic and pharmaceutical formulations.

### Example 2

### Synthesis of the Acyl Salicylate Derivatives from Acid Chlorides

1 Equivalent of salicylic acid was dissolved in 10 mL of a benzene—pyridine mixture (10.1.5 v/v) and 1 equivalent of the acid chloride was added dropwise. The reaction was left to proceed for 150 minutes and the acyl derivative was purified as described in Example 1.

### Example 3

### Evaluation of Heat Shock Response in NHEK Cells

Acetyl, propanoyl, n-butanoyl, n-octanoyl and n-decanoyl salicylates, synthesized according to Example 1 or 2, were tested for their ability to induce heat shock response in an in vitro system using cells NHEK.

NHEK cells, grown at 37° C. to 75% confluence, were treated with different doses of propanoyl, n-butanoyl, n-octanoyl and n-decanoyl salicylates at a concentration range from 0 to 50 μM, using 50 mM sorbitol and heat shock at 42° C. as positive controls. After incubation for 18 hours at 37° C., Hsp70 levels were quantitatively determined on keratinocytes using the ELISA kit by StressGen.

Propanoyl, n-butanoyl, n-octanoyl and n-decanoyl salicylates were found to increase Hsp70 levels in the keratinocytes. In particular:
5 μM propanoyl salicylate increased Hsp70 up to 89%
20 μM n-butanoyl salicylate increased Hsp70 up to 95%,
50 μM n-octanoyl salicylate increased Hsp70 up to 130%
50 μM n-decanoyl salicylate increased Hsp70 up to 140%.

### Example 4

### Evaluation of Heat Shock Response in L 929 Fibroblast Cells

Acetyl, propanoyl, n-butanoyl, n-octanoyl and n-decanoyl salicylates were tested for their ability to induce heat shock response in an in vitro system using L 929 fibroblast cells, in which the enzyme luciferase was cloned under the transcriptional control of the human Hsp70 promoter (Proc. Natl. Acad. Sci USA, 92, 7207-7211, 1995). The effects of the mentioned compounds on heat shock induction at different temperatures have been compared using Bimoclomol as reference compound (Table 1). As reported in Table 1, propanoyl salicylate and Bimoclomol at 37° C. increase the heat shock response by 33% with respect to the control, while n-butanoyl, n-octanoyl and n-decanoyl salicylates increase it up to 67%. Compared with the control, propanoyl salicylate at 40° C. increases the response by 107% and n-butanoyl salicylate by 70%. At this temperature, Bimoclomol increases the heat shock response by only 20%, whereas acetyl salicylate behaves as the control. The analysis of the data obtained shows that acyl-salicylates are able to induce heat shock response at lower temperatures than Bimoclomol, which shows maximum induction activity at 43° C. (Nature Medicine, 3, 1150-1154, 1997).

TABLE 1
Effect of the mentioned compounds on heat shock induction
at different temperatures in comparison with Bimoclomol.

Temperature stress

| | |
|---|---|
| 37° C. | 40° C. |

| | |
|---|---|
| Compounds | Luciferase activity (arbitrary units × 10⁻⁵) |

| | | |
|---|---|---|
| None | 0.3 | 4.6 |
| Acetyl salicylate | 0.4 | 4.8 |
| Propanoyl salicylate | 0.4 | 9.5 |
| Butanoyl salicylate | 0.5 | 8.0 |
| Octanoyl salicylate | 0.5 | 6.3 |
| Decanoyl salicylate | 0.5 | 6.5 |
| Bimoclomol | 0.4 | 5.5 |

### Example 5

### Protection of Fibroblasts from Cytotoxic Proteins

The protective effect of long chain acyl salicylates on NIH 3T3 mouse fibroblasts in the presence of cytotoxic protein aggregates, is tested, in comparison with the effects of acetyl salicylic acid and salicylic acid. The cytotoxid proteins employed are fibrillar aggregates obtained in vitro from the N-terminal acyl phosphatase-like domain of e. coli (HypF-N) according to Chiti et al. (Protein Sci. 10: 2541-2547, 2001). The noncytotoxic control is the soluble form of this protein. A positive response in this cellular model is indicative of a compound's ability to induce activation in the cell of the protein rescue pathway that, under stress conditions, reduces the cytotoxic effect of the protein aggregates.

Salicylic acid, acetyl salicylate and n-decanoyl salicylate, were tested at 5 and 50 μM doses, to determine the ability to induce a heat shock response. NIH 3T3 cells are grown at 37° C. for 24 hours, and then treated for 18 hours with the test compounds. After removal of the incubation medium, the cells are suspended for 24 hours in fresh medium containing cytotoxic levels of a protein aggregate or identical doses of the corresponding soluble protein. The cell viability after treatment is measured by the MTT method: any cell with mitochondrial activity incubated for 34 hours with MTT [3-(4,5-dimethyltriazole-2-yl)-2,5-diphenyl tetrazolium bromide] develops water insoluble formazan crystals, whereas dead cell don't produce them. With the subsequent addition of an organic solvent the formazan crystals are made soluble and their concentration, evaluation spectrophotometrically at 550-570 nm gives a measure of the cell vitality.

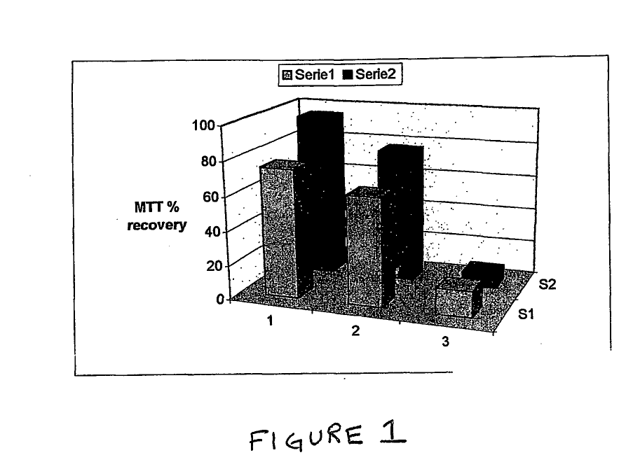

FIG. 1 shows that the most active compound in detoxification activity is decanoyl salicylate, which at a 5M dose gives an increase of 75% with respect to the control, while a 50 μM dose gives an almost complete protection (95% recovery of cell viability. Aspirin (acetyl salicylate) has a similar behavior but its protective effect is less pronounced. Salicylic acid, in contrast, is unable to protect cells from the cytotoxic effect of the protein aggregates.

### Example 6

### Prevention and Repair of Skin Damages by UV-B

The effect of acyl-salicylates in the prevention and repair of UV-B induced skin damage was determined with an in vivo test, on female Hartley albino guinea pigs, using the propanoyl salicylate as model compound. The animals, that weighed about 400 g, were shaved with a commercial cream 24 h before irradiation. Propanoyl salicylate was applied 120 minutes before irradiation on the head distal area, on a 5 cm² rectangular area at a dose of about 2 mg/cm², the head proximal area serving as control site. A bank of four lamps providing a mean irradiation of about 1.2 mW/cm² at 310 nm was used as UV-B source. Following light exposure, sites were occluded with a cotton pad. The erythema following UV-B exposure was evaluated according to the following scale: not irradiated, 0, pale pink; slight erythema, 1, pink; moderate erythema, 2, intense pink; severe erythema, 3, intense pink, edema; ulcerated erythema, 4, intense pink, ulceration. One MED (minimal erythemal dose to obtain an erythema grade 1) for untreated animals corresponds to 5 minutes exposure at 400 mJ/cm². The protection effect was calculated as the ratio of protected skin MED to unprotected skin MED. The erythema was observed in these conditions four hours after irradiation. The SPF (sun protection factor) of the propanoyl salicylate cream, prepared according to Example 7, is 4. Erythema appeared in the treated area only 7-10 hours after UV-B irradiation.

### Example 7

### Acceleration of Burns Healing

The effect of salicylic derivatives on wound healing was tested on rats. Partial to full thickness burns were created on rats skin by an electro-heating probe. Treatment of burns with 1% propanoyl salicylate in vaseline ointment significantly improved the wound closure compared to the control.

### Example 8

### Cosmetic Formulation

Concentrations are in % w/w.
Formulation A: deionized water 74.75; polysorbate 20 2.50; disodium ED TA 0.05; xanthan gum 0.20; propanoyl salicylate 5.00; butylene glycol 4.00.
Formulation B: light mineral oil 5.00; sorbitan palmitate 3.00; cetearyl octanoate 2.00; dimethicone 0.50, cocoa butter 0.80.
Formulation C: bisabolol 1.00; imidazolidinyl urea 0.50; Phenonip Nipa (phenoxyethanol, methylparaben, ethylparaben, propylparaben and butylparaben): 0.50; fragrance 0.20.

Procedure: keep A and B in separate vessels, heat to 75° C. and mix until compounds dissolve; add B to A at 75° C. under turbine agitation; add C to AB at 45° C. under turbine agitation for 20 minutes until smooth and lustrous.

What we claim is:

1. A method of inducing the production of heat shock proteins in a cell which comprises applying to the cell an induction-effective amount of a C₃-C₂₅ acyl salicylate.

2. The method of claim 1 in which the acyl salicylate is a C₃-C₁₂ acyl salicylate.

3. The method of claim 2 in which the acyl salicylate is selected from the group consisting of propanoyl salicylate, n-butanoyl salicylate, n-octanoyl salicylate, and n-decanoyl salicylate.

4. The method of claim 1 in which the cells are skin cells.

5. A method of treating or alleviating a disease condition associated with a heat shock response which comprises administering to an individual in need of such treatment or alleviation an induction effective amount of a C₃-C₂₅ acyl salicylate.

6. The method of claim 5 in which the condition is selected from the group consisting of cardiomyopathy, neuropathy, diabetes, ischemia, amyotropic lateral sclerosis, Alzheimer's disease, Parkinson's disease and Huntington's disease.

7. The method of claim 5 in which the acyl salicylate is a C₃-C₁₂ acyl salicylate.

8. A method of preventing, alleviating or treating damage to cells or tissues exposed to environmental stress which comprises administering to the cells or tissue an induction effective amount of a C₃-C₂₅ acyl salicylate.

9. The method of claim 8 in which the acyl salicylate is administered before exposure to the stress.

10. The method of claim 8 in which the stress is selected from the group consisting of UV exposure, tobacco smoke, air pollution, oxidative stress, and chemical exposure.

11. The method of claim 10 in which the stress is UV exposure.

12. The method of claim 8 in which the acyl salicylate is a C₃-C₂₅ acyl salicylate.

13. The method of claim 8 in which the cells are skin cells.

14. A method of treating, alleviating or preventing damage to the skin which comprises applying to the skin an induction effective amount of a C₃-C₂₅ acyl salicylate.

15. The method of claim 14 in which the damage is due to a wound.

16. The method of claim 14 in which the damage is due to photo- or chronoaging.

17. The method of claim 14 in which the acyl salicylate is a C₃-C₁₂ acyl salicylate.

18. A method of preventing or alleviating damage due to shock in living tissue or organs which comprises treating the tissue or organs with an induction effective amount of a C₃-C₂₅ acyl salicylate.

19. Pharmaceutical or cosmetic compositions comprising induction effective amounts of a C₃-C₂₅ acyl salicylate.

20. A composition for topical application to the skin comprising induction effective amounts of a C₃-C₂₅ acyl salicylate.
